(19) United States
(12) Patent Application Publication
(10) Pub. No.: US 2010/0170013 A1
(43) Pub. Date: Jul. 1, 2010

(54) Parga No. 2

(75) Inventor: Silvestre Arias, Fresno, CA (US)

Correspondence Address: MARK D MILLER;KIMBLE, MACMICHAEL & UPTON, 5260 NORTH PALM AVENUE, SUITE 221, FRESNO, CA, 93704, US

(73) Assignee: FOWLER PACKING COMPANY, INC., Fresno, CA (US)

(21) Appl. No.: 12/347,901

(22) Filed: Dec. 31, 2008

(51) Int. Cl. A01H 5/08

(52) U.S. Cl. 800/316

(57) ABSTRACT

A new distinct variety of tangerine characterized by producing a small to medium size fruit that is mature for harvesting and shipping in mid October about nine to ten days earlier than Clementine mandarin and with good flavor.

### BACKGROUND OF THE NEW VARIETY

In the fall of 2007, the inventor observed a single tree of Clementine mandarin (Citrus Reticulata Blanco) that was fully colored and mature nine to ten days earlier than the other Clementine mandarin trees in a 40-acre orchard in an area just south of the City of Fresno, Calif., in the San Joaquin Valley of Central Calif. This single tree on C-35/Citrange rootstock, Ponicrus Trifoiate×Citrus Sinensis, produced a heavy crop in the fourth year (2008) with the fruit varying in size from small to medium, yellow or orange color when fully ripe with sugar content of 11% at maturity, Oct. 14-18, 2008, with a pleasant flavor.

### Origin and Asexual Reproduction of the New Variety

The new variety of Citrus Reticulata Blanco is a bud sport of Clementine mandarin. The sport of Clementine mandarin was observed in 2007 because of its continuing earlier maturity and harvest than the original Clementine mandarin while maintaining its distinguishing characteristics.

### SUMMARY OF THE NEW VARIETY

The Parga No. 2 is characterized by producing a small to medium yellow-orange fruit with a pleasant flavor and good productive capacity that matures for harvesting and shipment October 14-16 in the Fresno area of the San Joaquin Valley. This variety can be compared to Clementine mandarin but matures nine to ten days earlier.

### BRIEF DESCRIPTION OF THE DRAWINGS

FIG. 1 is a color photograph of the new variety displaying a fruit attached to a twig and several leaves across the top and to the left followed on the right by a young branch with leaves attached displaying upper and lower surfaces.

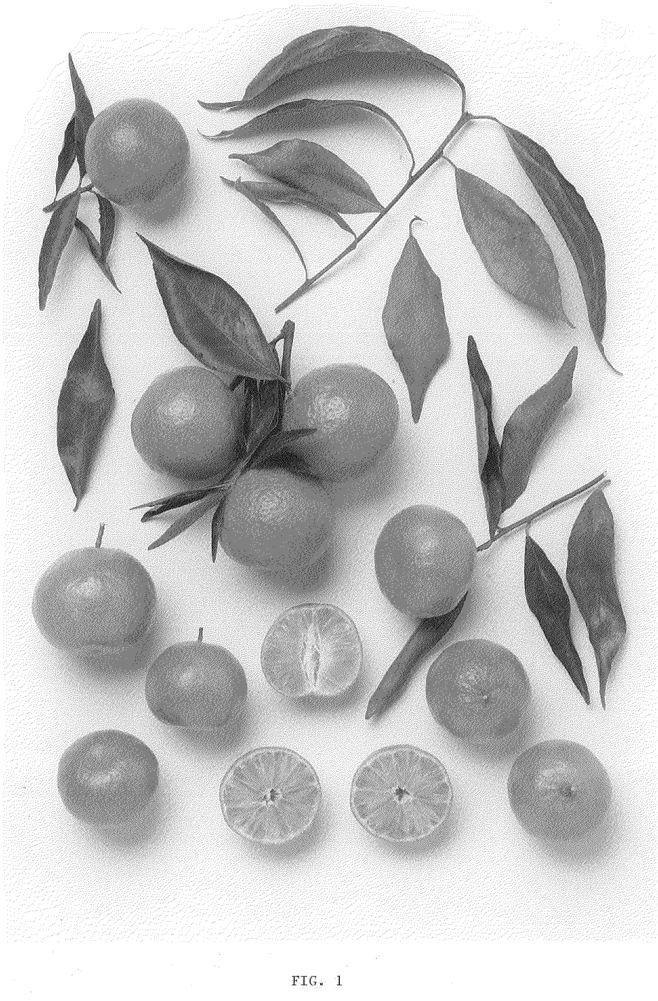

Directly below is a cluster of three fruits attached to a young branch with several leaves.

To the right is a side view of a fruit attached to a young branch and leaves.

Across the lower part of the photograph are two rows of fruit. On the upper row and to the left is a side view of fruit and stem attached, and to the right a smaller fruit. Then to the right is a fruit cut in half along center displaying the flesh followed by a bottom view of the fruit on the right.

On the bottom row is a side or lateral view of fruit followed by two halves displaying the segments of the flesh of the fruit. Finally, on the right is a fruit displaying the top view.

### DETAILED DESCRIPTION

Referring more to the horticultural description of the new and distinct variety of Mandarin tangerine, the following description has been observed under the ecological conditions prevailing at the origin orchard which is located near the City of Fresno in the San Joaquin Valley of Central Calif. All major color descriptions are by reference to the Dictionary of Color by Maerz & Paul, First Edition, published in 1930. Common colors are also employed.

### Tree

The new variety of the present application is generally medium in size.

Height: 8-10 feet (244-304 cm)

Width: 8 feet (244 cm)

Vigor: very good

Figure: upright with a spreading shape

Productivity: very good

Regularity of Bearing: regular

### Trunk

Size: 13 inch (33.02 cm) circumference, 8 inches (20.32 cm) above ground level, 4 inches (10.16 cm) above graft
Color: Mauve Taupe Copra (7-C-8)
Surface Texture: slightly rough

### Branches

Size: Medium, 3-4 inches (7.62-10.16 cm) in circumference at approximately 4 years
Bark Surface Texture: Smooth
Bark Color:

Mature: Mt. Vernon Green (23-J-8)

Immature (less than one year): Cossack green (23-J-11)

### Leaves

Size: large for Mandarin group
Length: 4.33-4.72 inches (11.00-12.00 cm)
Width: 1.46-1.89 inches (3.71-4.80 cm)
Shape: elliptic to oblong
Color:

Upper surface: Evergreen (24-E-12)

Lower surface: Art gr. (22-L-7)
Petiole: 0.39 inch (0.99 cm)

Petiole color: Cypress Green+Forest Green (23-L-6)
Marginal form: slightly serrated
Leaf Vein Color: Cosse Gr. (19-L-5)
Margin: Entire

Size: 0.24 inch (0.61 cm)
Numbers: Many
Surface Texture: smooth
Thickness: 0.08 inch (2 mm)
Stem Glands: none

### Flowers

Flower Buds:

Size: small, 0-2.00 inches, (0-5.08 cm)

Shape: conic

Surface texture: nearly smooth
Flowers generally showy
Date of First Bloom: April 25
Date of Full Bloom: May 7
Flowers:

Size: 0.79-0.91 inches (2.0-2.3 cm)

Petals: 4-5

Petal Color: White (1-A-1)

Petal Length: 0.16-0.20 inch (0.41-0.51 cm)

Petal Width: 0.08-0.11 inches (0.20-0.28 cm)
Pedicel:

Size: 0.14-0.20 inch (0.36-0.51 cm)

Color: Popcorn (9-J-3)
Number of sepals: 4-5

Color: Endive (17-L-3)
Stamens: 13-14
Filament color: White (1-A-1)
Anthers: Small

Color: Cavalry, Deep Chrome Y (9-L-7)
Pistil length: 0.28-0.31 inch (0.71-0.79 cm)

Color: Apricot Y. (9-K-5)
Fragrance: mild
Pollen color: yellow (1-A-10)

### Fruit

Date of Maturity: mid October
Size: small

Average height: 1.75 inch (4.45 cm)
Average width: 1.74 inch (4.42 cm)
Average weight: 3.91 ounces (110.85 grams)
Size: medium

Average height: 2.43 inches (6.17 cm)

Average width: 2.52 inches (6.40 cm)

Average weight: 4 ounces (113.40 grams)
Form: globose to oblong
Stem:

Length: 0.20 inch (0.51 cm)

Length: 0.19-0.20 inch (0.48-0.51 cm)

Color: Eden Gr (20-L-9)
Rind:

Color: Cadmium Y (9-L-8)

Thickness: average 0.08-0.16 inch (0.20-0.41 cm)

Surface: slightly pebbled to rugose
Segments: Flesh is 12-14 regularly separated segments

Vesicles: globose to obovoid

Flesh Color: Golden glow (9-L-6)

Skin: tender

Pulp Color: uniform Calvary Deep Chrome Y^{P} (9-L-7)
Juice:

Quantity: moderate

Color: Empire Y. (9-K-3)

Sugar: 11%

Sugar Acid Ratio: 12.0
Seeds: Seedless
Tree Storage: fruit hangs well on the tree
Use: Fresh market
Keeping quality: Excellent
Shipping Quality: Excellent
Resistance to disease: no disease observed

The above description of a new variety of Clementine mandarin is based on growing conditions in the Central San Joaquin Valley of Calif. Variations of the usual magnitude such as differences in maturity dates and production may be due to cultural activities such as irrigation, fertilization, pruning and climatic change.

What is claimed is:

1. A new distinct variety of tangerine characterized by producing a small to medium size fruit that is mature for harvesting and shipping in mid October about nine to ten days earlier than Clementine mandarin and with good flavor.
